(19) United States
(12) Patent Application Publication
(10) Pub. No.: US 2006/0134160 A1
(43) Pub. Date: Jun. 22, 2006

(54) Calcium phosphate coated implantable medical devices and processes for making same

(75) Inventors: Tomasz Troczynski, Vancouver (CA); Doma Hakimi, Vancouver (CA); Buhsung Hyun, Vancouver (CA); Mehrdad Keshmiri, Bumaby (CA); Mao-Jung M Lien, Maple Ridge (CA); Arc Rajtar, Coquitham (CA); Douglas Smith, Vancouver (CA); Pui H.M Tsui, Richmond (CA); Quanzu Yang, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: The University of British Columbia, Vancouver, BC (CA)

(21) Appl. No.: 10/527,406

(22) PCT Filed: Sep. 12, 2003
(86) PCT No.: PCT/CA03/01405

§ 371 Date: Nov. 18, 2005

Related U.S. Application Data
(60) Provisional application No. 60/410,307, filed on Sep. 13, 2002.

(51) Int. Cl. A61K 33/42; A61F 13/00; B05D 3/02

(52) U.S. Cl. 424/422; 427/2.1; 424/602

(57) ABSTRACT

This invention relates to novel calcium phosphate-coated implantable medical devices and processes of making same. The calcium-phosphate coatings are designed to minimize the immune response to the implant (e.g. restenosis in stenting procedures) and can be used to store and release a medicinally active agent in a controlled manner. Such coatings can be applied to any implantable medical devices and are useful for a number of medical procedures including (but not limited to) balloon angioplasty in cardiovascular stenting, ureteral stenting and catheterisation. The calcium phosphate coatings can be applied to a substrate as one or more coatings by a sol-gel deposition process, an aerosol-gel deposition process, a biomimetic deposition process, a calcium phosphate cement deposition process, an electro-phoretic deposition process or an electrochemical deposition process. The coating can contain and elude a drug in an engineered manner.

### FIELD OF THE INVENTION

This invention relates to novel calcium phosphate-coated implantable medical devices and processes of making same. The unique calcium-phosphate coated implantable medical devices minimize immune response to the implant. The coated implantable devices have the capability to store and release one or more medicinally active agents into the body in a controlled manner.

### BACKGROUND OF THE INVENTION

Cardiovascular stents are widely used in coronary angioplasty procedures to enlarge coronary arteries and thereby allow better blood circulation. Typically this is accomplished by a balloon angioplasty procedure wherein a contracted stent, usually in the form of a metallic mesh tube, is moved in to the site of blood vessel narrowing along a guide wire. Once the stent is in place an internally situated balloon expands it radially. After expansion the balloon is deflated and removed from vessel while the stent remains expanded in place. The stent thus provides a scaffold support for the walls of the blood vessel, enlarging the vessels aperture and increasing blood flow. This operation saves millions of lives annually around the world. Unfortunately the placement of metallic stents often leads to harmful side effects. A relatively large proportion of patients (up to half of the population, according to some statistics) experience an immune response to the implanted stent called inflammatory restenosis, and other negative effects, which lead to a re-narrowing of the vessel. This typically requires repeat surgical treatment within 1-2 years of the original balloon angioplasty operation.

The mechanisms that lead to restenosis and other immune responses associated with the implantation of a medical device are initiated by damage to the vessel lining during the surgical procedure. Such damage is very difficult to avoid entirely, but its effects, i.e. inflammation and/or infection, may be diminished through modifications to the surface of metallic implantable medical devices. The most common surface modification of implanted medical devices is the application of a thin polymer film coating. These coatings are frequently impregnated with medically active agent(s) such as antibiotics, anti-inflammatory agents and other, more complex drugs. These medically active agents are released from the coating through leaching to the arterial wall and the blood stream, often aided by dissolution of the carrier film. Typically, biodegradable polymers such as polylactic acid, polyglycolic acid, and others, frequently in combination with heparin and other anti-thrombogenic agents, are selected in such drug delivery systems. A particular advantage of the polymer coatings on stents is that the coatings are flexible and generally non-thrombogenic.

In the past, polymeric materials have been used for drug delivery control and have enjoyed substantial clinical success for certain drug systems. Unfortunately, even biodegradable polymers, although more bio-friendly than the native metallic surface, are still recognized by living tissue as foreign objects. Therefore the bio-degradation process is frequently accompanied by inflammatory response of the tissue. In some critical applications, such as cardiovascular stents, it has been determined that polymer coated stents do not perform according to expectations in longer term (in excess of 1 year) of use. Furthermore, in many instances relatively rapidly resorbing polymer coatings are quickly depleted, from the stent surface with concomitant loss of the long-term affects of the drug and harmful exposure of the bare metal surface to contact tissue. This may result in an adverse response of the tissue, leading to inflammation, restenosis (in the case of stents), and requiring repetitive surgical intervention.

There is therefore a strong need to discover materials for coating implantable medical devices that are entirely biocompatible and thus do not cause any adverse effects in the tissue. Furthermore, ideally this coating material will be able to deliver one or more pharmaceutically active agents to a targeted site. Studies have shown that porous coatings may accept the required load of drugs through adsorption and then release the drugs in a controlled manner. The drug release process is dependant on surface properties of the coating-material and the adsorption properties, molecular size, and other characteristics of the drug.

One group of materials exhibiting desired characteristics has been known for a long time, and is used extensively for the surface modification of large rigid implants such as artificial hips in the human body. These materials are members of the family of calcium phosphates (CaP) and include hydroxyapatite (HA), di- and tri-calcium phosphates, as well as partially or fully amorphous calcium phosphates. These materials are mineral components of hard tissue and as such are fully bio-compatible and bio-resorbable with no side effects. Calcium phosphate, in particular hydroxyapatite (HA), is a principal inorganic component of bone, and thus offers entirely new perspectives for coating-based drug encapsulation and drug delivery systems.

Hydroxyapatite ceramics, Ca₁₀(PO₄)₆(OH)₂, belong to the class of calcium phosphate (CaP) based bioactive materials that are used for a variety of biomedical applications, including matrices for drug release control [M. Itokazu et al., Biomaterials, 19, 817-819, 1998; F. Minguez et al Drugs Exp. Clin. Res., 16[5], 231-235, 1990; W. Paul and C. P. Sharma, J. Mater. Sci. Mater. Med., 10, 383-388, 1999]. Other members of the CaP family, such as dicalcium phosphate (CaHPO₄.2H₂O) or tricalcium phosphate (Ca₃(PO₄)₂), have also been used for similar purposes. The CaP family of materials has been long recognized as having a high degree of biocompatibility with human tissue.

The use of calcium phosphate coatings, including HA coatings, thermally deposited on implantable devices has been limited by the fact that such coatings used to date have had thicknesses of >0.01 mm and have exhibited brittle behaviour when in bulk form. This characteristic has limited their use to applications where a solid support structure, such as dental or hip implant, does not allow for much deformation of the structure. In such cases, the potential for coating damage is limited and osseo-integration with the tissue occurs in an improved manner. HA coated implants in particular have been shown to possess excellent biocompatibility and provide accelerated integration of the implant with the surrounding tissue. The bio-resorption rate of such coatings can be controlled through adjustment of their crystallinity and chemical composition, e.g. by the incorporation of carbonate groups and other methods known to those skilled in the art.

A method alternative to thermal coating is the biomimetic deposition of HA films at room temperature (BM-HA). This technique has been used for a variety of biomedical applications, for example drug delivery [H. B. Wen et al, J. Biomed. Mater. Res., 41, 227-36, 1998; S. Lin and A. A. Campbell, U.S. Pat. No. 5,958,430, 1999; D. M. Liu et al J. Mater. Sci. Mater. Med., 5, 147-153, 1994; K. de Groot et al, J. Biomed. Mater. Res., 21, 1375-1381, 1987). This forming mechanism is driven by supersaturation of Ca²⁺ and PO₄³⁻, under appropriate solution pH, where HA is the most stable phase. As the process proceeds at or near room temperature, the apatitic crystals which form through nucleation and growth may incorporate biologically active species, such as antibiotics, anti-cancer drugs, anti-inflammatory agents, etc. The deposition rates for BM-HA are in the range of 0.05-0.5 μm/h.

This relatively low deposition rate may be enhanced significantly if electric field is applied to the metallic substrate being coated, e.g. stent, in a solution containing proper concentration of calcium and phosphorous ions. This variant of coating is usually referred to as Electro-Chemical Deposition (ECD), and the resulting film termed as ECD-HA. As ECD also proceeds at (or near) room temperature, drug encapsulation is also possible in ECD-HA. The physiological solutions for BM-HA formation are naturally water-based, which makes it impossible to encapsulate hydrophobic bioactive agents into BM-HA coatings. The biomimetic HA films (both BM-HA and ECD-HA) may be deposited on implantable medical devices at room temperature, which is of great advantage for drug encapsulation during deposition.

Unfortunately, the bonding strength BM-HA and ECD-HA to metallic surfaces is generally significantly lower than that of sol-gel HA (termed here SG-HA). At the same time, bonding strength of BM-HA or ECD-HA to previously consolidated hydroxyapatite is high, generally in excess of 40 MPa. In this respect building additional BM-HA or ECD-HA film on top of the already existing, well-bonded to the metallic substrate film of SG-HA provides a novel and inventive route to achieve high bonding strength, controlled porosity, and drug encapsulation capability of the films deposited at room temperature,

Another alternative for room (or near-room) temperature deposition of porous calcium phosphate films, in particular hydroxyapatite, for drug impregnation and encapsulation, is so-called calcium phosphate cement (CPC) route. In this previously disclosed process (refer to U.S. Patent Application No. US2002/0155144 A1 “Bifunctional Hydroxyapatite Coatings and Microspheres for in-situ Drug Encapsulation”, by T. Troczynski, D. Liu, and Q. Yang), fine particles of calcium phosphate precursor Ca(OH)2 and calcium phosphate salt monocalcium phosphate anhydrate, are milled and mixed in ethanol, followed by film deposition and impregnation by sodium phosphate solution (refer to the Example 4 below for details of this procedure). As a result of this process, microporous, semi-amorphous CPC-HA results, suitable for delivering drugs through leaching and during film resorption. Similarly as above, CPC-HA film bonds poorly to metallic surfaces, such as those of implants or stents. However, CPC-HA film deposited on previously consolidated surface of HA, such as SG-HA, achieves high bonding strength, generally in excess of 40 MPa. In this respect building additional CPC-HA film on top of the already existing, well-bonded to the metallic substrate film of SG-HA provides a novel and inventive route to achieve high bonding strength, controlled porosity, and drug encapsulation capability of the films deposited at room temperature.

Electric field-assisted thin film deposition technologies have the great advantage of the resulting film uniformity, especially for complex substrates such as stents. One such technology termed Electro-Phoretic Deposition (EPD) is well known method in ceramic processing. In this method fine particles of a ceramic (generally about a micrometer or less in size) suspended in a liquid attain electric charge through interaction with the liquid or through addition to the suspension of surface-active species. The simplest example of such EPD system is oxide (or hydroxide, such as hydroxyapatite) ceramic powder suspended in water and acid (such as nitric acid) mixture. In such environment protons will have a tendency to absorb on surface of the ceramic particles, providing positive charge to the particles. Upon application of electric field, such charged particles would migrate to the negative electrode (cathode). Exactly opposite would happen in basic environment, i.e. negatively charged particles of ceramic would migrate to the positive electrode (anode). EPD is an excellent technique for deposition of ceramic films, including calcium phosphate films, as disclosed in U.S. Pat. No. 5,258,044, dated Nov. 2, 1993 (“Electro-phoretic Deposition of Calcium Phosphate Material on Implants”, by D. D. Lee). Unfortunately, EPD films must be sintered at relatively high temperature to gain sufficient structural integrity. For example, the EPD films of calcium phosphate disclosed in U.S. Pat. No. 5,258,044, had to be sintered at between 600° C. and 1350° C. These temperatures are high enough to induce substantial change to the metallic substrate, e.g. in terms of surface oxidation or microstructural changes (e.g. grain growth).

Drug encapsulation in HA has been achieved in the past by simple post-impregnation of a sintered, porous HA ceramic [K. Yamamura et al, J. Biomed. Mater. Res., 26, 1053-64, 1992]. In this process, the drug molecules simply adsorb onto the surface of the porous ceramic. The drug release is accomplished through desorption and leaching of the drug to the surrounding tissue after exposure to physiological fluid. Unfortunately, most of the adsorbed drug molecules release from such system in a relatively short period of time. Impregnation of drug material into porous sintered calcium phosphate microspheres has been reported in the patent literature. “Slow release” porous granules are claimed in U.S. Pat. No. 5,055,307 [S. Tsuru et al, 1991], wherein the granule is sintered at 200-1400° C. and the drug component impregnated into its porosity. “Calcium phosphate microcarriers and microspheres” are claimed in WO 98/43558 by B. Starling et al [1998], wherein hollow microspheres are sintered and impregnated with drugs for slow release. D. Lee et al. [WO98/16209] claim poorly crystalline apatite wherein macro-shapes harden and may simultaneously encapsulate drug material for slow release. It has been suggested to use porous, composite HA as a carrier for gentamicin sulfate (GS), an aminoglycoside antibiotic to treat bacterial infections at infected osseous sites [J. M. Rogers-Foy et al, J. Inv. Surgery 12 (1997) 263-275]. The presence of proteins in HA coatings did not affect the dissolution properties of either calcium or phosphorus ions and that it was solely dependent on the media [Bender S. A. et al. Biomaterials 21 (2000) 299-305].

Stents are disclosed in several patent publications. U.S. patent publication No. 2002/0007209 A1, published Jan. 17, 2002, de Sheerder et al., discloses an expandable metal tube prosthesis with laser cuts in the walls. The prosthesis can be coated with titanium nitride (TiN) for bio-compatibility. The holes in the walls of the prosthesis can be used to locally administer medicines and the like.

U.S. Pat. No. 6,387,121 B1, issued May 14, 2002, Alt, assigned to Inflow Dynamics Inc., discloses a stent constructed with a tubular metal base. The stent can be constructed to have three layers (see FIG. 2). The first layer 15 is typically 316L stainless steel. The intermediate layer 50 is formed of a noble metal or an alloy thereof, preferably selected from a group consisting of niobium, zirconium, titanium and tantalum (see column 7, lines 58-61). The third or outer layer 80 is preferably composed of a ceramic-like metal material such as oxide, hydroxide or nitrate of metal, preferably iridium oxide or titanium nitrate, as a bio-compatible layer that serves as a primary purpose to avoid tissue irritation and thrombus formation.

EP 0 950 386 A2, published Oct. 20, 1999, Wright et al., assigned to Cordis Corporation, discloses a thin walled stent which is formed as a cylinder with a plurality of struts. The struts have channels formed therein. Therapeutic agents can be deposited in the channels. Rapamycin specifically is mentioned as a therapeutic agent which can be deposited in the channels to prevent restenosis (re-narrowing) of an artery.

### SUMMARY OF THE INVENTION

The invention is directed to an implantable medical device with a calcium phosphate coating comprising: (a) substrate; and (b) calcium phosphate coating on the substrate, said coating having desired bonding and porosity characteristics.

The calcium phosphate coating of the device can be hydroxyapatite. The thickness of the calcium phosphate coating can be between about 0.00001 mm and 0.01 mm, and preferably about 0.001 mm to 0.0001 mm. The tensile bond strength between the substrate and the calcium phosphate coating can be greater than about 20 MPa. The calcium phosphate coating can be deposited on the device as particles having a diameter between about 1 μm and 100 μm and a thickness of between about 1 μm to 10 μm. The particles can cover about 20% to about 90% of the surface of the substrate.

The implantable medical device can be constructed of stainless steel, cobalt alloy, titanium cobalt-chromium or metallic alloy. The calcium phosphate coating can be porous and the pores can retain a drug. The rate of release of the drug from the pores can be controlled in an engineered manner.

The substrate can have a first calcium phosphate coating and a second calcium phosphate coating and the drug can be contained in both the first and the second coating or only in one coating. The drug can be one which inhibits restenosis. The calcium phosphate coating can be dicalcium phosphate, tricalcium phosphate or tetracalcium phosphate. The device can be a human or animal tissue implantable device. The device can be a stent which is coated with calcium phosphate.

The invention is also directed to a process of coating an implantable medical device with a calcium phosphate coating comprising: (a) hydrolyzing a phosphor precursor in a water or alcohol based medium; (b) adding a calcium salt precursor to the medium after the phosphite has been hydrolyzed to obtain a calcium phospate gel; (c) depositing the calcium phosphate gel as a coating on the surface of a substrate; and (d) calcining the calcium phosphate coating at a suitable elevated temperature and for pre-determined time to obtain a crystallized calcium phosphate having desired crystallinity, bonding and porosity characteristics.

The deposition of the coating on the substrate can be performed by aerosol deposition, dip-coating, spin-coating, electrophospate coating or electrochemical coating. The calcium phosphate coating can be calcined at a temperature of at least about 350° C. The calcium phospate gel can be hydroxyapatite gel.

The porosity of the calcium phosphate coating can be controlled and can retain a drug. The rate of release of drug can be controlled. The calcium phosphate coating can be hydroxyapatite, dicalcium phosphate, tricalcium phosphate or tetracalcium phospate.

The phosphate precursor can be an alkyl phosphite or a triethyl phosphate. The calcium precursor can be a water-soluble calcium salt. The water soluble calcium salt can be calcium nitrate.

The invention is also directed to a process of coating a soft tissue implantable device with a calcium phosphate coating comprising: (a) providing a soft tissue implantable substrate; (b) depositing a calcium phosphate coating on the substrate utilizing a biomimetic deposition process; or (c) depositing the calcium coating on the substrate utilizing a calcium phosphate cement deposition process; or (d) depositing the calcium phosphate coating on the substrate utilizing an electro-phoretic deposition process; or (e) depositing a calcium phosphate coating on the substrate utilizing an electrochemical deposition process.

The device can be a calcium phosphate coated stent. The calcium phosphate coating can be hydroxyapatite. The calcium phosphate coating can be deposited discontinuously on the substrate as discrete particles.

A first calcium phosphate coating can be deposited on the substrate utilizing an aerosol-gel process, a sol-gel process or an electro-phoretic deposition process or an electro-chemical deposition process and a second calcium phosphate coating can be deposited on the first coating or the substrate utilizing an aerosol-gel process, a sol-gel process, a biomimetic process, a calcium phosphate cement process, an electro-phoretic deposition process or an electrochemical deposition process.

The calcium phosphate coating can contain and elude a drug. The calcium phosphate coating can be coated with a hydrogel film. The calcium phosphate can be deposited on the substrate as discontinuous non-equiaxial particles. The non-equiaxial particles can have an average size of about 0.1 μm and a thickness up to about 0.01 mm. The first and second coatings can contain a drug.

The ratio of calcium to phosphate in the sol-gel precursor can be engineered to enable various phosphate phases to be obtained. The calcium phosphate phase can be hydroxyapatite, dicalcium phosphate, tricalcium phosphate or tetracalcium phospate.

### DRAWINGS

In drawings which illustrate specific embodiments of the invention, but which should not be construed as restricting the spirit or scope of the invention in any way:

FIG. 1A is a micrograph of a stainless steel (316L) stent coated with discontinuous ASG-HA thin film.

FIG. 1B is a magnification of the sector indicated by the rectangle of FIG. 1A.

FIG. 2A is a micrograph of a stainless steel stent (316L) coated with discontinuous ASG-HA thin film and crimpled, with no damage to the coating.

FIG. 2B is a micrograph of the same stent as shown in FIG. 2A after expansion showing no damage to the coating.

FIG. 3A is a micrograph of a stainless steel (316L) stent coated with continuous EPD-HA thin film.

FIG. 3B is an about 4×6 μm magnification of the sector indicated by the rectangle of FIG. 3A.

FIG. 4A is a micrograph of a stainless steel (316L) stent coated with continuous ECD-HA thin film.

FIG. 4B is an about 65×88 μm magnification of the sector indicated by the rectangle of FIG. 4A.

### DETAILED DESCRIPTION OF THE INVENTION

Throughout the following description specific details are set forth in order to provide a more thorough understanding of the invention. However, the invention may be practiced without these particulars. In other instances, well known elements have not been shown or described in detail to avoid unnecessarily obscuring the present invention. Accordingly, the specification and drawings are to be regarded in an illustrative, rather than a restrictive, sense.

The invention in one embodiment is directed to implantable medical devices with a flexible thin film calcium phosphate bio-compatible and bio-resorbable coating that has the ability to act as a high capacity drug carrier. Such CaP coatings have no side-effects during coating dissolution into body fluids, and can be designed with a high level of control of coating dissolution rate and microstructure, which also determine the drug retention and release characteristics.

Of all the types of implantable medical devices that exist, the coronary stents utilized in balloon angioplasty procedures provide a useful model for testing the effectiveness of sol-gel deposited thin flexible CaP coatings on such stents due to the fact that such stents are designed to be flexible. The use of such stents in the examples below should not, however, be considered as limiting the application of the CaP coatings described only to stents. The invention has broad application to virtually any type of body implantable device.

We have determined unexpectedly that the intrinsic brittle behaviour of CaP ceases to limit the system strain capability if the strongly bonded coating is sol-gel deposited and is thinner than approximately 0.001 mm. Experiments involving repeated contraction/expansion of such thin CaP sol-gel coated stents reveal that there is no separation of the coating from the stent, nor visible damage to the coating, if the coating is thinner than about 0.001 mm and is strongly bonded to the substrate (the tensile bond strength should be larger than about 40 MPa, as measured in model strength experiments according to ASTM C-633 standard).

In addition, we have discovered that if the novel sol-gel process for deposition of calcium phosphates, in particular hydroxyapatite (HA) synthesis (as previously disclosed in our U.S. Pat. No. 6,426,114 B1, Jul. 30, 2002, “Sol-Gel Calcium Phosphate Ceramic Coatings and Method of Making Same”, by T. Troczynski and D. Liu) is used, the resulting thin flexible coating has controlled porosity which may be utilized to retain drugs within the coating, and release the drugs at a controlled rate.

The invention pertains to a sol-gel (SG) process for synthesis of calcium phosphate, in particular, hydroxyapatite (HA), thin film coatings on implantable medical devices. The process allows the HA to be obtained in a controlled crystallized form, at a relatively low temperatures, i.e. starting at ≈350° C. This is an unexpectedly low crystallization temperature for HA sol-gel synthesis. The process provides excellent chemical and physical homogeneity, and bonding strength of HA coatings to substrates. The low process temperature avoids substrate metal degradation due to thermally-induced phase transformation, microstructure deterioration, or oxidation.

Disclosed herein is a method wherein uniform films of hydroxyapatite by the electro-phoretic deposition (EPD) method (EPD-HA) are deposited on complex stent surface, and there is no need to pursue sintering in excess of 500° C. to achieve substantial structural integrity of the film and its high bonding strength to the metallic substrate. In this method, the first step is the well-known EPD of the HA film, for example as disclosed in U.S. Pat. No. 5,258,044, using suspension of sub-micrometer particles of HA in water. This film is dried and then heat treated at 500° C. for 10-60 minutes to initiate sintering of HA. The film is still too weak and too poorly bonded for practical use as a coating on stent or other medical device or implant, but is sufficiently strong to survive the subsequent processing step comprising impregnation by aero-sol-gel HA droplets. The droplets penetrate porosity of the previously deposited EPD-HA, strongly aided by the capillary suction. Thus, majority of the pores of the EPD-HA film are penetrated by the sol-gel precursor of HA, all the way to the metallic substrate. This composite film can be now dried and sintered at a relatively low temperature or 400-500° C., due to the very high activity of the sol-gel component of the film. The sol-gel film bonds the particles of HA deposited by EPD, and bonds well to the metallic substrate during the heat treatment Thus, both the film uniformity (due to EPD process) and low-temperature sinterability (due to sol-gel process) have been achieved. This novel and inventive hybrid technology for uniform HA coatings on stents has the ability to produce films in thickness range from about 1 micron to above 100 microns, with porosity in the range from about 10 vol % to about 70 vol %. Such porous thick HA films are excellent carriers for drugs loaded through impregnation into open porosity of the film. Details of such hybrid process, and its several variants, for preparation of HA films on stents, are given in the examples below.

Problems with drug delivery in vivo are frequently related to the toxicity of the carrier agent, the generally low loading capacity for drugs, and the aim to control drug delivery resulting in self-regulated, timed release. With the exception of colloidal carrier systems, which support relatively high loading capacity for drugs, most organic systems deliver inadequate levels of bioactive drugs. Sol-gel films heat-treated at relatively low temperatures closely resemble the properties of colloidal films, in terms of accessible surface area and porosity size.

The sol-gel process according to the invention allows the calcium phosphate to be obtained in a crystallized form, at relatively low temperature, i.e. approximately 350-500° C. Variation of the heat treatment temperature and time provides for control of coating crystallinity (i.e. a more amorphous, more easily resorbable coating can be processed at lower temperatures) as well as coating porosity (higher porosity and smaller average pore size at lower temperatures). Variation of Ca/P ratio in the sol-gel precursor mix allows one to obtain various calcium phosphate phases, for example, hydroxyapatite, dicalcium phosphate, tricalcium phosphate or tetracalcium phosphate.

The invention in one embodiment is directed to a sol-gel process for preparing calcium phosphate, such as hydroxyapatite, which comprises: (a) hydrolysing a phosphor precursor in a water or alcohol based medium; (b) adding a calcium salt precursor to the medium after the phosphite has been hydrolysed to obtain a calcium phosphate gel such as a hydroxyapatite gel; (c) depositing the gel on the surface of an implantable medical device; and (d) calcining the calcium phosphate, such as hydroxyapatite, at a suitable elevated temperature and for pre-determined time to achieve desired crystallinity, bonding and porosity characteristics for the coating on the device. The deposition of the gel can be done by any number of methods, such as aero-sol deposition, dip-coating, spin-coating, electrophoretic deposition.

In a preferred embodiment, the phosphor precursor can be an alkyl phosphite and the alkyl phosphite can be triethyl phosphite. Further the calcium precursor can be a water-soluble calcium salt and the water soluble calcium salt can be calcium nitrate. The crystallized calcium phosphate can be calcined at a temperature of at about 350° C. or higher. The metallic implantable medical device can be stainless steel, cobalt alloy, a titanium substrate or other metallic alloy substrate.

We have discovered that if certain specific characteristics of the calcium phosphate coatings are maintained, the coatings become highly flexible while maintaining their chemistry, high bio-compatibility, and bio-resorbability. The most important characteristics are (a) coating thickness, and (b) the strength of the coating bonding to the metallic substrate. We have repeatedly demonstrated (refer to the examples below) that if CaP coating thickness is maintained below about 0.001 mm, and its bonding strength to the metallic substrate is above approximately 40 MPa, the substrate-coating system retains the strain capabilities of the substrate alone, i.e. the system maintains its integrity during deformation.

Furthermore, we have discovered that thicker CaP coatings deposited discontinuously on metallic substrate, i.e. in the form of separate “islands” and “patches” approximately 1-100 μm in diameter, retain high resistance against substrate deformation. Our experiments have shown that stents coated with such 1-100 μm patches, about 1-10 μm thick, can be crimped and then expanded without damage to the patches of ceramic. These patches can be deposited on the substrate through a variety of methods discussed above, such as BM-HA, ECD-HA, CPC-HA (all at room or near-room temperature), or EPD-HA, SG-HA and combinations thereof (these two techniques including heat treatment at elevated temperatures). These coating deposition techniques are illustrated in the following examples. The discontinuous CaP film coated medical implant may have some fraction of an area of the metallic substrate exposed to living tissue, which may again lead to the adverse tissue reaction described above. This problem can be avoided by combining discontinuous CaP films with a continuous bio-compatible and non-thrombogenic polymer. Thus, a composite CaP-polymer coating on medical implant is the result. Furthermore, a thin (<0.001 mm) continuous CaP coating can be combined with a thicker discontinuous CaP coating.

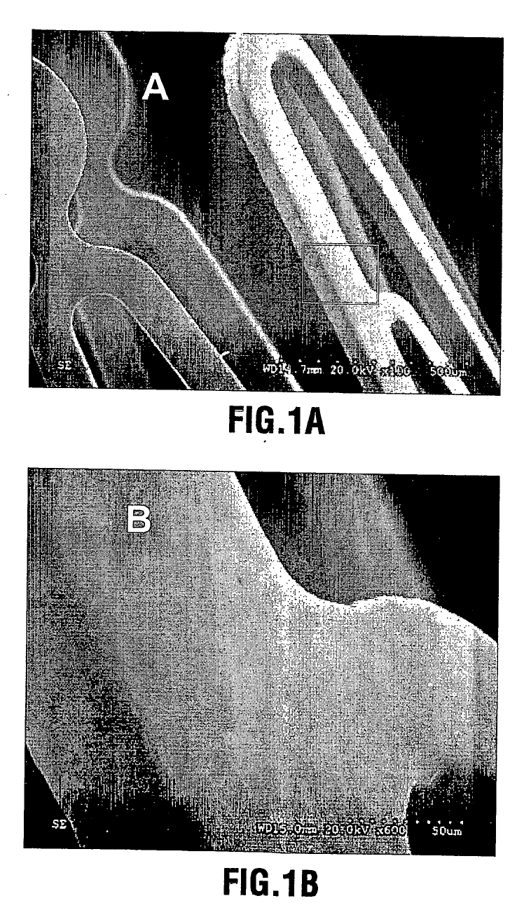

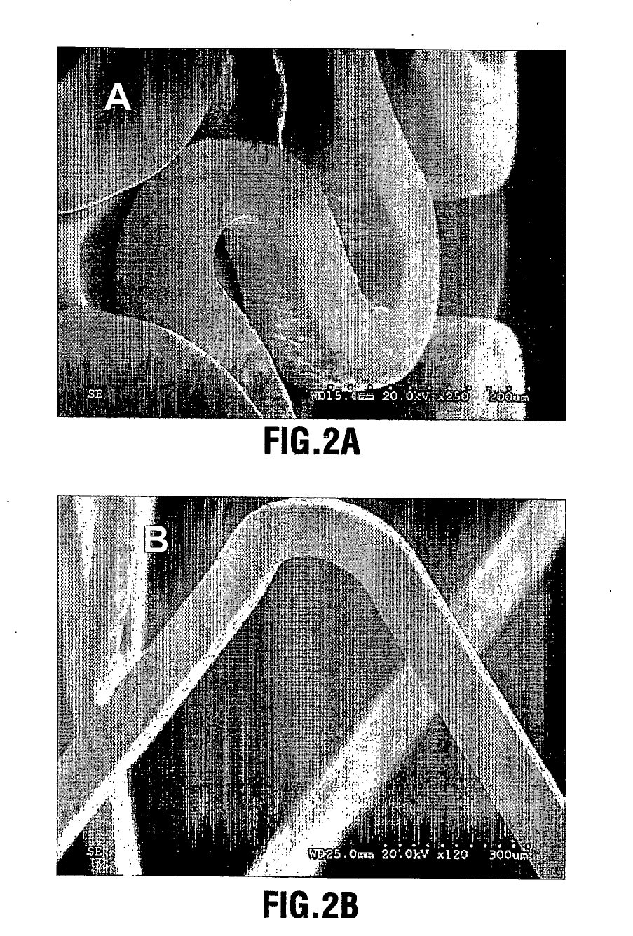

The effects of this process (described in detail in the Examples) are shown in the representative FIGS. 1 and 2. FIG. 1A illustrates stainless steel (316L) stent coated with discontinuous ASG-HA thin film; FIG. 1B is a magnification of the sector of (A) indicated by the rectangle. FIG. 2A illustrates a stainless steel (316L) stent coated with discontinuous ASG-HA thin film and crimped, with no damage to the coating. FIG. 2B is the same stent after expansion, showing no damage to the coating.

Our discovery of flexible continuous/discontinuous CaP films or CaP/polymer films opens up a range of new applications of highly biocompatible Cap coatings for medical implants, particularly, but not limited to those that require deformation capability such as coronary stents.

A sol-gel (SG) process provides superior chemical and physical homogeneity of the final ceramic product compared to other routes, such as solid-state synthesis, wet precipitation, or hydrothermal formation. The SG process allows the desired ceramic phase, e.g. thin film CaP coating, to be synthesized at temperatures much lower than some of the alternate processes. In the SG coating process substrate metal degradation due to thermally induced phase transformations and microstructure modification or oxidation, is avoided. SG widens green-shaping capability, for example, and it is a very convenient method for deposition of thin ceramic coatings.

Sol-Gel deposition of HA (SG-HA) films at elevated temperatures (350-500° C.) was disclosed previously in U.S. Pat. No. 6,426,114 B1. Sol-gel (SG) processing of HA allows molecular-level mixing of the calcium and phosphor precursors, which improves the chemical homogeneity of the resulting calcium phosphate. The crystallinity of the calcium phosphate phase can be enhanced by appropriate use of water treatment during processing. Variation of Ca/P ratio in the sol-gel precursor mix allows one to obtain any of a number of calcium phosphate phases, for example, hydroxyapatite, dicalcium phosphate, tricalcium phosphate or tetracalcium phosphate. The versatility of the SG method provides an opportunity to form thin film coatings, either continuous or discontinuous, in a rather simple process of dip-coating, spin-coating or aero-sol deposition.

A high degree of HA crystallinity is frequently required for longer-term bioactive applications, because partially crystalline, or amorphous calcium phosphate, such as HA, coatings are rapidly resorbed by living tissue. For the presently disclosed application of thin HA films on implantable medical devices, control of crystallinity of the HA coating is possible through variation of the time/temperature history during processing. This allows control of the coating resorption rate and thus rate of release of the drugs impregnated into microporosity of the coating.

Ceramics produced by sol-gel processing can be designed to include high fraction of pores, with well-defined (narrowly distributed) pore size. This is a consequence of the chemical route to the final oxide ceramic produced through SG. Only a small fraction of the original precursor mass is finally converted to the ceramic oxide, the remaining fraction being released during heat treatment, usually in the form of gas, is usually as a combination of water and carbon dioxide. Thus, the released gases leave behind a large fraction of porosity, up to 90% in some instances, depending on the drying conditions and heat treatment time and temperature. These pores can be as small as several nm in diameter, again depending on the drying conditions and heat treatment time and temperature. Effectively, the accessible surface area of such sol-gel derived oxide ceramics can reach several hundred square meters per gram of the oxide, making it an excellent absorbent of gas or liquid substances, or solutions. For example, the average pore size in sol-gel HA treated at relatively low temperature of 400° C. is about 5 nm in diameter, with 90% of pore diameters falling within the range of 1-30 nm. This unique porosity characteristic is widely utilized to produce desiccants, filters and membranes of sol-gel derived ceramic. In this respect sol-gel derived ceramic oxides have a great advantage over polymers, which are in general difficult to process to possess high porosity and high accessible surface area. In the present invention, we utilize this unique property of sol-gel derived CaP coatings on medical implants, especially stents, possessing high accessible surface area to make it a high-capacity drug carrier.

In the text of this application, it is understood that when appropriate, the term “calcium phosphate” (CaP) is used generically and includes minerals such as hydroxyapatite, dicalcium phosphate, tricalcium phosphate, tetracalcium phosphate and amorphous or partially amorphous calcium phosphate. Studies on the sol-gel route to thin film calcium phosphate coatings on implantable medical devices, particularly stents, performed by the inventors have led to an unexpected break-through in process development. The method according to the invention has produced CaP coatings after heat treatment in air, starting at about 350° C. We have unexpectedly discovered that the film is highly flexible if it is thinner than about 0.001 mm, thereby allowing damage-free manipulation of a CaP coated deformable implantable medical device, for example the contraction and expansion of a CaP coated stent. Preferably, the coating has a thickness between about 0.0001 and 0.001 mm. Furthermore, in this application, we have discovered that the film can accept drugs into its fine porosity, thereby allowing it to address the adverse phenomena related to common medically implanted devices, i.e. the restenosis that occurs after placement of a coronary stent in a blood vessel.

The calcium phosphate coating according to the invention has been deposited on stents and other metallic surfaces using variety of techniques, including dip-coating, spin-coating, aero-sol deposition electrophoretic deposition. The coatings were deposited on stents made of 316L stainless steel and tubes, and on other metallic substrates including cobalt-iron alloy and titanium.

### EXAMPLES

To demonstrate the feasibility of the unique processing concepts outlined above, the following examples are described below for stainless steel substrate and coronary stents. The procedures outlined below can be applied to other implantable medical devices.

### Example 1

In the first stage of the process, phosphite sol was hydrolysed in a water-ethanol mixture (a concentration of 3M) in a sealed beaker until the phosphite was completely hydrolysed (which is easily recognized by loss of a characteristic phosphite odour), at ambient environment. A Ca salt (2M) was then dissolved in anhydrous ethanol, and the solution was then rapidly added into the hydrolysed phosphite sol. The sol was left at ambient environment for 8 hours, followed by drying in an oven at 60° C. As a result of this process, a white gel was obtained. For the sol containing Ca/P ratio required to produce HA, the gel showed a pure (single phase) apatitic structure with a Ca/P ratio of 1.666, identical to stoichiometric HA, after calcining at a temperature as low as 350° C. Varying the Ca/P ratio allows other calcium phosphates, such as dicalcium phosphate (Ca/P=1) or tricalcium phosphate (Ca/P=1.5), to be obtained. A coating produced using this process, and applied to 316 SS substrate, showed adhesive strength of about 40 MPa after curing at a temperature<450° C. The coating was crack-free and porous.

### Example 2

In another variant of the process, a pure water-based environment was used. The aqueous-based sols were prepared in the same manner as described above in Example 1 for the ethanol-based system. A higher rate of hydrolysis of the phosphite sol was observed. The mixed sol was dried while stirring. After 8 hours aging, a white gel appeared. For the sol containing a Ca/P ratio required to produce HA an apatitic structure with Ca/P ratio of 1.663, close to stoichiometric HA, resulted after calcining the gel at a temperature of 350° C. Both the ethanol-based and aqueous-based gels showed essentially the same apatitic structure at relatively low temperatures. This invention provides a method of synthesizing the HA ceramics via an aqueous-based sol-gel process.

### Example 3

A CaP coating was deposited on the surfaces of a group of electropolished stainless steel stents through aerosol-gel processing. The stents were first treated in 2.4 N phosphoric acid solution for 10 minutes at 70° C. to clean the surface and produce microroughness for increased bonding of the coating. The treated stents were ultrasonically cleaned and dried. The CaP sol was prepared by (a) hydrolysing a phosphor precursor (phosphite); (b) adding a calcium salt precursor to the medium after the phosphite has been hydrolysed to obtain a calcium phosphate sol such as a hydroxyapatite sol. The sol was atomized into ^{˜}4 μm large particles using ultrasonically assisted atomizer, and the resulting aerosol fed into a coating chamber. This specific deposition technique is referred to as Aero-Sol-Gels (ASG) deposition and the resulting hydroxyapatite film as ASG-HA.

The clean stent was inserted into the coating chamber filled with flowing CaP aerosol-gel for a period of 30 seconds, while maintaining the aerosol flow at 0.1 liter/min and chamber temperature at 50° C. The temperature of the coating chamber affects the deposition mode of the coating, producing a uniform, film like coverage of the surface as evidenced by SEM. The coating was dried at 60° C. and heat treated at 450° C. for 15 min to crystallize CaP to form hydroxyapatite thin film. The procedure produces a thin coating covering uniformly the surface of the stent. The thickness of the coating is measured using ellipsometry in the range of 50-150 nm. The subsequent SEM studies on the crimped and expanded coated stents show no evidence of cracking or delamination of the coating. This proves the reliability of the uniform, thin continuous CaP coating during the deployment and implantation of the stent into the coronary artery.

### Example 4

CaP coating has been deposited on the surface of an electropolished stainless steel stents through aerosol-gel processing (ASG), as described in Example 3. The chamber temperature was maintained at 25° C. The coating was dried at 60° C. and heat treated at 450° C. for 15 min to crystallize CaP to form hydroxyapatite thin film. The procedure explained above produces a coating comprising of isolated island of approximately 2-6 μm in size and 0.1-2 μm in thickness, scattered uniformly on the surface of the stent, and covering about 70% of the surface of the stent, as shown in FIGS. 1A and 1B. Subsequent SEM studies on the crimped and expanded coated stents showed no evidence of cracking or delamination of the coating, as shown in FIGS. 2A and 2B. This proves the reliability of the discontinuous CaP coating of variable thickness during the deployment and implantation of the stent into the coronary artery.

### Example 5

Stainless steel metallic substrates (316L) were coated with a 0.6-0.8 μm thin layer of apatite (ASG-HA) as described in Example 3. One group of samples was annealed at 400° C. for 20 min to achieve crystalline SG-HA(C) film and another group at 375° C. for 60 min to achieve amorphous SG-HA(A) film. These films were used as nucleation site for precipitation of BM-HA film. The SG-HA coated samples were immersed into “simulated body fluid” (SBF) of ionic composition (in units of mmol/l) 142 Na⁺, 5.0 K⁺, 2.5 C²⁺, 1.5 Mg²⁺, 103 Cl⁻, 25 HCO₃⁻, 1.4 HPO₄²⁻, and 0.5 SO₄²⁻. The SBF was buffered at pH 7.4 with tris(hydroxymethyl)-aminomethane and HCl. This in-vitro static deposition (i.e. the SBF was not renewed during the deposition period) at ˜24° C. produced good quality, dense 3-5 μm thick BM-HA film deposits on flat SG-HA substrates. The crystalline SG-HA(C) film is coated with dense BM-HA, whereas amorphous SG-HA(A) film is coated with porous BM-HA. The properties of the underlying SG-HA surface modification film can be used to vary the properties, e.g. porosity, of the nucleated and deposited top BM-HA film for drug encapsulation.

### Example 6

Stainless steel metallic stents (316L) were coated with −0.1 μm thin CaP coatings as described in Example 3. An inorganic colloidal slurry containing calcium phosphate precursor Ca(OH)₂ and calcium phosphate salt monocalcium phosphate anhydrate, was ball milled in ethanol. The two starting inorganic ingredients had particle size 0.3-2 μm and 0.5-4 μm, respectively. The initial Ca/P ratio in the slurry was kept at 1.5. As dissolution and precipitation are the principal mechanisms for apatite development in such system, 5 wt % of submicron, crystalline hydroxyapatite powder was used as seeds for heterogeneous nucleation of CPC-HA. The thin CaP film surface-modified sample was dip coated in the ethanol suspension of the precursors. After single dip coating, an approximately 10 μm thick layer of porous precursor powder mixture developed on the substrate due to rapid evaporation of ethanol. Due to the colloidal nature of the precursors slurry, this film develops sufficient structural integrity (i.e. strength and hardness) to accept the next processing step. In this step, the film is exposed to sodium phosphate water-based solution (0.25 M), which is allowed to soak into the open pores of the film, and then placed in an incubator at 37° C., 100% relative humidity, for 24 h. During incubation, the colloidal precursors react with the phosphate liquid and precipitate HA. In order to assess the possibility of using this double-coating route for controlled drug release, amethopterin (Sigma Chemicals, USA) was employed as a model drug, in an amount of 5% based on solid phase content of CPC-HA precursors. The drug was mixed with the colloidal suspension of the precursors, before dip coating was performed. During incubation period, 20 μm thick CPC-HA coating precipitated encapsulating the drug molecules within the nanopores of the crystallizing HA. After encapsulation, a drug release study was conducted by immersion of the substrates into 20 ml of phosphate buffer saline (PBS, pH=7.4) at constant ratio of (CPC coating weight)/(volume of PBS) of 1 mg/ml. A reference sample coated with hydrogel film was also tested for drug release kinetics. The hydrogel film was prepared by dipping the CPC-HA layer containing the drug into a polymer solution containing 3% polyvinyl alcohol. After drying, the weight gain of the ˜20 mg CPC-HA layer due to the additional hydrogel coating was ˜0.5 mg, corresponding to the content of polymer film in the CPC-HA matrix of about 2.5%. The samples of PBS liquid with released drug were periodically taken out (i.e. entire liquid was emptied) and refilled with the same amount of 20 ml of PBS. The drug concentration in the supernatant was determined via an UV-Visible spectroscopy. Although a burst effect was detected for both coatings over the initial period of about 8 h, a slower release is evident for the sample post-coated with hydrogel. A linear relationship was obtained between the amount of drug released and (time)^{1/2} for the release time greater than 8 h.

### Example 7

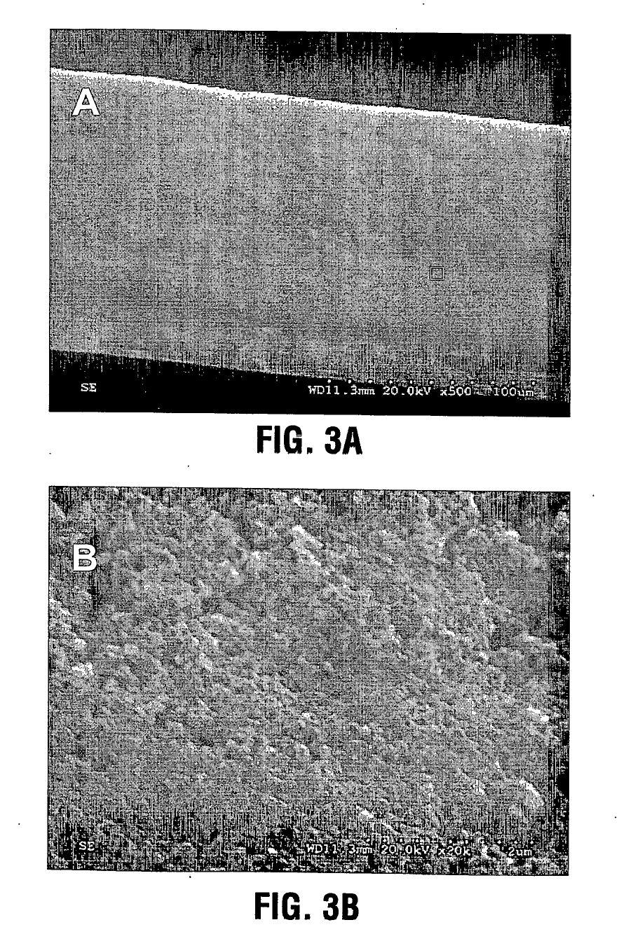

The stent was submerged into water-based, diluted suspension of sub-micron particles of hydroxyapatite, containing approximately 2 wt % of HA in the suspension. DC voltage of 5V was applied to the stent, for times varying from 5 seconds, to 10 minutes. As the particles of HA naturally attain positive charge in such solution, they are attracted to the stent surface which is also a negative electrode (cathode) in this system. The buildup of HA particles attracted to the stent (cathode) allows to produce an extremely uniformly coated surface, thickness of the coating varying as a function of time of application of voltage. The film uniformity is the biggest advantage of such Electro-Phoretic Deposition (EPD) processing, which is difficult to reproduce using other methods such as sol-gel processing. For the short time of 10 sec., the EPD-HA coating thickness is about 1 micrometer. This type of EPD-HA coating on 316L stainless steel stent is illustrated in FIG. 3. For the longer times of several minutes, the coating thickness may exceed 10 micrometers. Thus, in this EPD process, a controlled thickness, uniform HA film may be produced. The as deposited film constitutes loosely bonded particles of HA, of porosity generally in excess of 50 vol %. In order to increase structural integrity and bonding strength to the substrate of such EPD film, heat treatment is necessary at temperatures at least 500° C., for times at least 10 minutes. The heat treatment of EPD films proceeds at higher temperatures and longer times than sol-gel films, because HA particles deposited in the EPD process are less reactive than those deposited in the sol-gel process. The goal of such heat treatment is to increase interparticle bonding, while providing sufficient residual porosity to maintain low stiffness and flexibility of the film, and to provide room for drug impregnation. The need for higher temperature and longer times heat treatment of EPD films is a disadvantage, as the heat treatment process may adversely affect properties of the metallic substrate of the stent.

### Example 8

The HA was deposited on a 316L stainless steel stent surface through EPD process as described in the Example 7. The uniformly deposited EPD film was heat treated at 500° C. for 10 minutes to achieve minimal structural integrity of the film, sufficient to survive handling and preventing re-fluxing of the film upon contact with liquid medium. Such EPD-coated stent was exposed to droplets of sol in the aero-sol-gel process described in Example 3. The sol droplets have penetrated open porosity of the EPD film, and, by capillary attraction, located themselves mostly within negative curvature of the necks between EPD deposited HA particles. Such composite coating was heat treated again at 500° C. for 10 minutes. Now the active sol-gel component of the coating allowed achieving high structural integrity of the film, while EPD component of the coating allowed achieving high uniformity of coverage by the film. A uniform, porous HA film was achieved in this novel combined process.

### Example 9

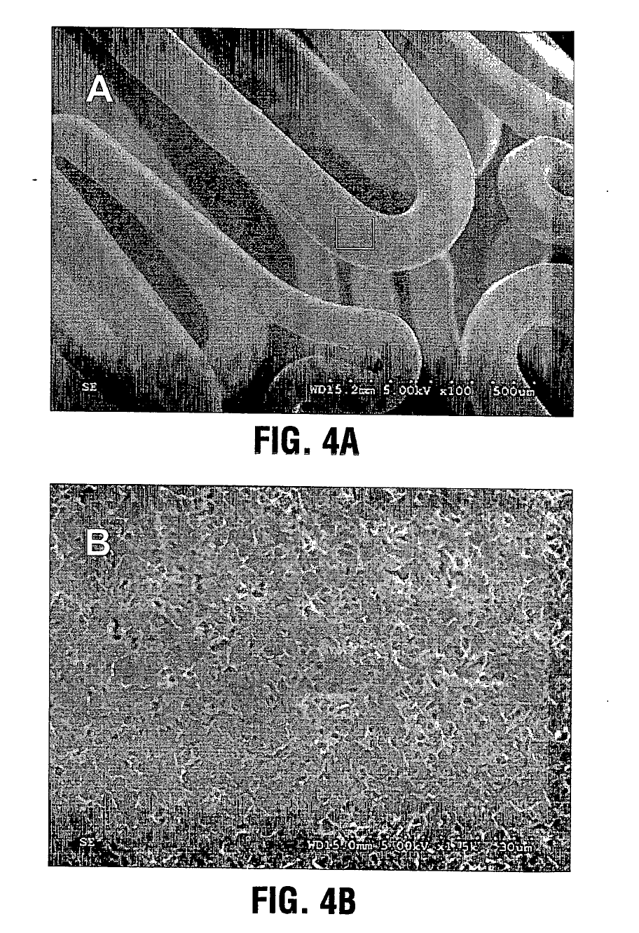

The electrochemical deposition (ECD) of hydroxyapatite HA has been conducted in the mixed aqueous solution of Ca(NO₃)₂ 4H₂O and NH₄—H₂PO₄. In this process HA is deposited on the cathodic (negatively biased) surface of stent or implant by the following reaction: 10Ca²⁺+6PO₄³⁻+2OH→Ca₁₀(PO₄)₆(OH)₂ ECD was conducted in the mixed aqueous solution of 0.02329 M Ca(NO₃)₂4H₂O and 0.04347 M NH₄H₂PO₄. The stainless steel specimen, i.e. stent, was the cathode, and platinum was used as the anode. The pH was controlled at 4.0 with the addition of sodium hydroxide. The environment temperature was controlled at 40° C.±1° C. The coating morphology deposited at low current density (1 mA/cm²) was a thin uniform porous structure, 1-2 micrometers thick for deposition time of 0.5-1 minute, as illustrated in FIG. 4.

### Example 10

The HA was deposited on a 316L stainless steel stent surface through ASG-HA process as described in the Example 4. The discontinuous network of HA patches left some of the stent surface uncoated. 5V DC bias voltage was applied to such pre-coated stent, and the stent submerged into suspension of submicron HA particles. The uncoated metallic surface of the stent preferentially attracted HA particles leading to preferential electrophoretic deposition (EPD) of HA in these areas, to build the coating about 1 micrometer thick in about 10 seconds. The coated stent was heat treated at 500 C for 10 minutes. The EPD-HA coated areas show increased porosity as compared to ASG-HA coated areas, suitable for impregnation with drug carrying liquid. Such composite engineered HA coating shows unique properties regarding mechanical performance and drug release properties.

### Example 11

The HA was deposited on a 316L stainless steel stent surface through ASG-HA process as described in the Example 3, followed by the process of ECD-HA deposition as described in Example 9, but on top of the already heat treated ASG-HA. Such composite engineered coating allowed to achieve substantially higher bonding strength (as compared to ECD-HA deposited directly on metallic surface), and capability of drug encapsulation during deposition of ECD-HA on top of ASG-HA.

### Example 12

The HA was deposited on two 316L stainless steel stents surface through ASG-HA process as described in the Example 4. The coated stents were evaluated in the standard thromboresistance test in dogs. Minimal thrombosis with a grade of 1 (defined as thrombus found at one location only) was observed in one out of two test sites. In the second test site, no thrombosis (grade 0) was observed.

The process for coating of calcium phosphate, in particular HA, bioactive ceramics, on implantable medical devices disclosed herein offers the following advantages in comparison to other processes and other coating materials on implantable medical devices:
(1) The coating process, including CaP sol synthesis, can be completed in ambient environment (i.e. air), in less than 24 hours.
(2) The thin (<0.001 mm) adhesive CaP coatings exhibit sufficient flexibility to survive substantial strain, e.g. during crimping and expanding of a coated stent, without coating damage or spallation
(3) Porous CaP coatings can be produced, with controlled amount and size of the pores, which allows design flexibility in choice and absorption/release characteristics for the drug impregnated into the coating
(4) The synthesis requires low temperature (˜350° C.) and short time (<1 hour) of calcination for formation of high quality, highly adhesive CaP coating. Low temperature calcination of the novel CaP coatings on metals permits thermal treatment in an air environment without the risk of metal oxidation and possible property degradation due to microstructural deterioration or phase transformations.

It will be clear for the person skilled in the art of sol-gel processing that coating deposition parameters, such as time, the flow rate of the aerosol, temperature of the coating chamber or the concentration of the sol-gel solution can be customized for different implantable medical device materials and applications producing various degree of coverage on the surface. Similar manipulation and optimization of process parameters may be applied to other coating methods disclosed, i.e. dip- and spin-coating and electrophoresis, biomimetic coating, electrochemical deposition coating, calcium phosphate cement coating, electrophoretic deposition coating, as well as coating porosity distribution and ratio of the inorganic phase (CaP) to organic phase (biodegradable polymer). These parameters were optimized for the particular CaP coatings on the implantable medical devices described in the foregoing examples.

It is well known that crystallinity and microporosity of hydroxyapatite directly affects its dissolution rate in body fluids. Different heat treatment regimes and temperatures can be adopted to produce various degrees of crystallinity and microporosity to control the degradation of the coating into the body environment. This advantage is of a great importance where drug delivery capabilities are added to the implantable medical device surface coated with sol-gel derived CaP. Similar deposition process can be applied to coating other metallic surfaces, such as Ti substrates or other alloys, such as Cobalt-Chromium-Nickel-Molybdenum-Iron. A thin uniform thin HA coating is obtained. The results of this experiment provide basic evidence of the feasibility of the as described coating on implantable medical devices composed of non-metallic materials such as polymers.

The nature of the process for CaP coatings deposition according to the invention is such that it can be easily incorporated into the current production practice of metallic implantable medical devices. The water-based liquid precursors to CaP ceramic coatings, simple deposition technique (e.g. dipping or spin-coating or aerosol deposition or electrophoretic deposition, and others) and low-temperature heat treatment in air make the process not unlike simple painting-curing operation which can be commercialized with relatively small effort.

As will be apparent to those skilled in the art in the light of the foregoing disclosure, many alterations and modifications are possible in the practice of this invention without departing from the scope thereof. Accordingly, the scope of the invention is to be construed in accordance with the substance defined by the following claims.

What is claimed is:

1. An implantable medical device with a calcium phosphate coating comprising:
(a) substrate; and
(b) calcium phosphate coating on the substrate, said coating having desired bonding and porosity characteristics.

2. A device as claimed in claim 1 wherein the calcium phosphate coating is hydroxyapatite.

3. A device as claimed in claim 1 wherein the thickness of the calcium phosphate coating is between about 0.00001 mm and 0.01 mm.

4. A device as claimed in claim 1 wherein the thickness of the calcium phosphate coating is between about 0.001 mm and about 0.0001 mm.

5. A device as claimed in claim 1 wherein the tensile bond strength between the substrate and the calcium phosphate coating is greater than about 20 MPa.

6. A device as claimed in claim 1 wherein the calcium phosphate coating is deposited on the device as particles having a diameter between about 1 μm and 100 μm and a thickness of between about 1 μm to 10 μm.

7. A device as claimed in claim 1 wherein the particles cover about 20% to about 99% of the surface of the substrate.

8. A device as claimed in claim 1 wherein the substrate is constructed of stainless steel, cobalt alloy, titanium cobalt-chromium or metallic alloy.

9. A device as claimed in claim 1 wherein the calcium phosphate coating is porous and the pores retain and elude a drug.

10. A device as claimed in claim 9 wherein the rate of release of the drug from the pores is controlled in an engineered manner.

11. A device as claimed in claim 10 wherein the substrate has a first calcium phosphate coating and a second calcium phosphate coating and the drug is contained in the first and second coatings.

12. A device as claimed in claim 9 wherein the drug inhibits restenosis.

13. A device as claimed in claim 1 wherein the calcium phosphate coating is dicalcium phosphate, tricalcium phosphate or tetracalcium phosphate.

14. A device as claimed in claim 1 wherein the device is a human or animal tissue implantable device.

15. A device as claimed in claim 14 wherein the device is a stent.

16. A process of coating an implantable medical device with a calcium phosphate coating comprising:
(a) hydrolyzing a phosphor precursor in a water or alcohol based medium;
(b) adding a calcium salt precursor to the medium after the phosphite has been hydrolyzed to obtain a calcium phospate gel;
(c) depositing the calcium phosphate gel as a coating on the surface of a substrate; and
(d) calcining the calcium phosphate coating at a suitable elevated temperature and for pre-determined time to obtain a crystallized calcium phosphate having desired crystallinity, bonding and porosity characteristics.

17. A process as claimed in claim 16 wherein the deposition of the coating on the substrate is performed by aerosol deposition, dip-coating, spin-coating, electrophosphate coating or electrochemical coating.

18. A process as claimed in claim 16 wherein the calcium phosphate coating is calcined at a temperature of at least about 350° C.

19. A process as claimed in claim 16 wherein the calcium phosphate gel is hydroxyapatite gel.

20. A process as claimed in claim 16 wherein the thickness of the calcium phosphate coating on the substrate is between about 0.00001 mm and 0.01 mm.

21. A process as claimed in claim 16 wherein the thickness of the calcium phosphate coating is between about 0.0001 mm to about 0.001 mm.

22. A process as claimed in claim 16 wherein the tensile bond strength between the calcium phosphate coating and the substrate is greater than about 20 MPa.

23. A process as claimed in claim 16 wherein the calcium phosphate gel is deposited on the substrate as particles having a diameter between about 1 μm and 100 μm.

24. A process as claimed in claim 16 wherein the porosity of the calcium phosphate coating is controlled and retains and eludes a drug.

25. A process as claimed in claim 24 wherein the rate of release of drug is controlled in a defined manner.

26. A process as claimed in claim 16 wherein the calcium phosphate coating is hydroxyapatite, dicalcium phosphate, tricalcium phosphate or tetracalcium phospate.

27. A process of coating a soft tissue implantable device with a calcium phosphate coating comprising:
(a) providing a soft tissue implantable substrate;
(b) depositing a calcium phosphate coating on the substrate utilizing a biomimetic deposition process; or
(c) depositing the calcium coating on the substrate utilizing a calcium phosphate cement deposition process; or
(d) depositing the calcium phosphate coating on the substrate utilizing an electro-phoretic deposition process; or
(e) depositing a calcium phosphate coating on the substrate utilizing an electrochemical deposition process.

28. A process as claimed in claim 27 wherein the substrate is a stent.

29. A process as claimed in claim 27 wherein the calcium phosphate coating is hydroxyapatite.

30. A process as claimed in claim 27 wherein the calcium phosphate coating is deposited discontinuously on the substrate as discrete particles.

31. A process as claimed in in claim 27 wherein a first calcium phosphate coating is deposited on the substrate utilizing an aerosol-gel process, a sol-gel process, an electro-phoretic deposition process or an electrochemical deposition process and a second calcium phosphate coating is deposited on the first coating or the substrate utilizing an aerosol-gel process, a sol-gel process, a biomimetic process, a calcium phosphate cement process, an electro-phoretic deposition process or an electrochemical deposition process.

32. A process as claimed in claim 27 wherein the calcium phosphate coating contains a drug.

33. A process as claimed in claim 27 wherein the calcium phosphate coating is coated with a hydrogel film.

34. A process as claimed in claim 27 wherein the calcium phosphate is deposited on the substrate as discontinuous non-equiaxial particles.

35. A process as claimed in claim 34 wherein the non-equiaxial particles have an average size of about 0.1 μm and a thickness of up to about 0.01 mm.

36. A process as claimed in claim 31 wherein both the first and the second coatings contain a drug.
